(19) United States
(12) Patent Application Publication
(10) Pub. No.: US 2004/0006120 A1
(43) Pub. Date: Jan. 8, 2004

(54) Use of histamine h3 receptor inverse agonists for the control of appetite and treatment of obesity

(76) Inventors: Stephen L Yates, Aurora, OH (US); Clark E Tedford, Russell, OH (US); Kurt R Brunden, Aurora, OH (US)

Correspondence Address: WOOD, PHILLIPS, KATZ, CLARK & MORTIMER, 500 W. MADISON STREET, SUITE 3800, CHICAGO, IL, 60661, US

(21) Appl. No.: 10/344,963

(22) PCT Filed: Aug. 15, 2001
(86) PCT No.: PCT/US01/41737

(51) Int. Cl. C07D 233/54; A61K 31/4172

(52) U.S. Cl. 514/400; 548/335.1

(57) ABSTRACT

A method for the use of histamine II₃ receptor inverse agonists in the regulation of appetite and treatment of obesity is disclosed. Presently preferred inverse agonists are imidiazole derivatives.

### FIELD OF THE INVENTION

The present invention is directed to a method for the use of histamine H₃ receptor inverse agonists in the regulation of appetite and treatment of obesity. Presently preferred inverse agonists are imidazole derivatives.

### BACKGROUND OF THE INVENTION

Obesity can be described as a state of excessive accumulation of body fat and is widely considered to be a major public health problem, associated with substantially increased morbidity and mortality, as well as psychological problems, reduced economic achievement and discrimination. Examples of health problems thought to be caused or exacerbated by obesity include coronary heart disease, stroke, obstructive sleep apnea, diabetes mellitus, gout, hyperlipidemia, osteoarthritis, reduced fertility, impaired psychosocial function, reduced physical agility and increased risk of accidents, and impaired obstetrical performance.

Causes of obesity remain unclear. However, whether obesity is of genetic origin or is promoted by a genotype-environment interaction, or both, it is evident that energy intake must have exceeded metabolic and physical (work) energy expenditure for there to have been surplus energy available for fat deposition. Considerable uncertainty remains concerning the relative importance of different mechanisms in achieving this positive energy balance.

Treatment of obesity is difficult. Although it is well-established that morbidity and mortality are increased in obese individuals, it is unclear whether dieting results in decreased long-term risk of early death. The major obesity intervention has been the many different forms of dieting, which are often fads without a sound scientific basis. Results have shown that dieting is a component of the weight loss regimens of 84% of women and 76-78% of men attempting to lose weight. A further important obesity intervention is physical activity which increases energy expenditure, both during the actual period of exercise and during the subsequent period of rest. Thus, exercise can promote negative energy balance, provided that energy intake is not increased concomitantly. Exercise, however, has been found to be only moderately successful in promoting weight loss. A program combining both dieting and exercise as well as behavior modification is widely viewed as the optimal approach to weight loss. Food restriction alone can be very successful in promoting weight loss, but a significant component of the weight loss can be lean tissue. In addition, food restriction results in a decline in total energy expenditure, which serves to reduce the extent of negative energy balance. Studies have demonstrated that combination programs involving both food restriction and exercise promote a substantial loss of fat and, at the same time, promote maintenance of lean tissue.

It is therefore evident that obesity is a problem, and that no reliable treatment thereof has been established. There is a continuing need to develop drugs and treatment regimes effective in the alleviation of obesity. We have now unexpectedly found that a certain group of compounds, histamine H₃ receptor inverse agonists, are particularly advantageous for use in the treatment of obesity.

Histamine (2-(4-imidazolyl)ethylamine) is found naturally in most tissues of both plants and animals. It exerts its biological actions by combining with cellular receptors located in or on the surface membrane. There are at least three distinct types of receptors: H₁, H₂ and H₃. Some of the known effects of histamine are exerted on smooth muscle and cardiac muscle, on endothelial and nerve cells and on the secretory cells of the stomach.

The histamine H₃ receptor is the latest receptor to have been identified. Stimulation of this receptor with H₃ receptor antagonists has been employed in animal models of central nervous system disorders, psychiatric disorders, sleep disorders and eating disorders. The majority of compounds synthesized for this purpose are derivatives of histamines, in other words, 2-substituted imidazoles.

For example, H₃ receptor antagonists have been disclosed in WO 96/40126; WO 96/38142 and in U.S. Pat. Nos. 5,990,317; 6,008,240 and 5,652,258. Moreover, certain H₃ receptor antagonists have been disclosed to have utility as appetite suppressants in U.S. Pat. No. 5,486,526.

Additionally, non-imidazole alkylamine histamine H₃ receptor antagonists have been disclosed as having utility for the treatment of obesity in EP 0 982 300 A2. Phenyl-alkyl-imidazole histamine H₃ receptor antagonists have been disclosed as having utility for the treatment of obesity in U.S. Pat. Nos. 5,990,147 and 6,034,251.

Thioperamide is a histamine H₃ receptor antagonist which has been disclosed as possibly affecting food intake in rats under certain conditions by Itoh et al., in “Thioperamide, a histamine H₃ receptor antagonist, suppresses NPY-but not Dynorphin A-induced feeding in rats”, Regulatory Peptides, 75-76 (1998) 373-376. Thioperamide was postulated to have differential affinity for the various conformations of the H₃ receptor by Clark et al., in “Differential effect of sodium ions and guanine nucleotides on the binding of thioperamide and clobenpropit to histamine H₃-receptors in rat cerebral cortical membranes” British Journal of Pharmacology, (1995) 114, 357-362. Such differential affinity is characteristic of inverse agonism. Similarly, thioperamide and burimamide were disclosed as compounds which discriminated between two classes of sites on H₃ receptors by West et al. in “Identification of Two H₃-Histamine Receptor Subtypes”, Molecular Pharmacology (1990) 38: 610-613. In addition to thioperamide, the antagonists GT-2212 and GT-2016 (5-cyclohexyl-1-(4-imidazol-4-yl-piperidyl)pentan-1-one) were also postulated to be potential inverse agonists by Tedford et al., in “Development of trans-2-(1H-imidazol-4-yl)cyclopropane Derivatives as New High-Affinity Histamine H₃ Receptor Ligands”, The Journal of Pharmacology and Experimental Therapeutics, 289: 1160-1168, 1999.

An inverse agonist is a ligand that preferentially stabilizes the inactive conformation of a G-protein coupled receptor. Only a few inverse agonists for the histamine H₃ receptor have been tentatively identified. As of yet, there has been no allusion as to the connection between the identification of a compound as a histamine H₃ receptor inverse agonist and the use of that compound for any particular treatment.

We have now discovered that certain histamine H₃ receptor antagonists are inverse agonists, and these inverse agonists can be used selectively to treat obesity through suppression of appetite, though other histamine H₃ receptor antagonists have no effect on appetite suppression.

### BRIEF SUMMARY OF THE INVENTION

The invention is directed to a method for promoting weight loss and treating eating disorders comprising administering to a patient in need of such weight loss or treatment an effective amount of an inverse agonist of histamine H₃ receptors, with the proviso that said inverse agonist is not thioperamide. Presently preferred inverse agonists for appetite suppression are 4-{(1R,2R)-trans-2-[O-(2-cyclohexylethyl)carboxamido]cyclopropyl}imidazole, 4-{(1R2R)-trans-2-[O-(2-cyclohexylmethyl)carboxamido]cyclopropyl}imidazole and 3-(1H-imidazol-4-yl)propyl-di(p-fluorophenyl)-methyl ether. The inverse agonist may be administered by intravenous, intramuscular, intraperitoneal or subcutaneous injection; or orally. From about 0.01 mg/kg to about 200 mg/kg of the inverse agonist may be administered in a single dose or divided dose per day.

### DETAILED DESCRIPTION OF THE INVENTION

The present invention is directed to methods for the use of histamine H₃ receptor inverse agonists in the regulation of appetite and treatment of obesity. Presently preferred inverse agonists are imidazole derivatives.

### Definitions of Terms

The term treatment, as used herein includes prophylaxis as well as alleviation of established obesity. In addition to the treatment of obesity, a method according to the present invention has application in the treatment of conditions associated with obesity, such as coronary heart disease, stroke, obstructive sleep apnea, diabetes mellitus, gout, hyperlipidemia, osteoarthritis, reduced fertility, impaired psychosocial function, reduced physical agility and increased risk of accidents, and impaired obstetrical performance. The ability of the inverse agonists to suppress appetite is the basis for their use in the treatment of obesity, for a decrease in the desire to eat will lead to a decrease in actual food intake, promoting weight loss if the patients' activity level remains the same or increases.

A recognized clinical and epidemiological measure for the classification of obesity is the Body Mass Index (BMI) which is defined as weight in kilograms divided by the square of height in meters. Typically, a BMI of 25-30 is considered as overweight and greater than 30 as obese. Treatment according to the present invention generally refers to a lowering of BMI to less than about 29 to 31. It will however be appreciated by persons skilled in the art that obesity is inherently difficult to classify, and that the cut-off point for the definition of obesity is necessarily arbitrary, in part because body fatness is a continuum. However, in general terms, treatment according to the present invention desirably prevents or alleviates obesity to an extent where by there is no longer a significant health risk to the patient.

As used herein, the term “composition” is intended to encompass a product comprising the specified ingredients in the specified amounts, as well as any product which results, directly or indirectly, from a combination of the specified ingredients in the specified amounts.

The inverse agonists of the present invention can be used in the form of pharmaceutically acceptable salts derived from inorganic or organic acids. The phrase “pharmaceutically acceptable salt” means those salts which are, within the scope of sound medical judgement, suitable for use in contact with the tissues of humans and lower animals without undue toxicity, irritation, allergic response and the like and are commensurate with a reasonable benefit/risk ratio. Pharmaceutically acceptable salts are well-known in the art. For example, S. M. Berge et al. describe pharmaceutically acceptable salts in detail in J. Pharmaceutical Sciences, 1977, 66: 1 et seq. The salts can be prepared in situ during the final isolation and purification of the compounds of the invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphor sulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methane sulfonate, nicotinate, 2-naphthalene sulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

Basic addition salts can be prepared in situ during the final isolation and purification of inverse agonists of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylammonium, dimethylammonium, trimethylammonium, triethylammonium, diethylammonium, and ethylammonium among others. Other representative organic amines useful for the formation of base addition salts include ethylenediamine, ethanolamine, diethanolamine, piperidine, piperazine and the like.

Dosage forms for topical administration of the inverse agonists of this invention include powders, sprays, ointments and inhalants. The active compound is mixed under sterile conditions with a pharmaceutically acceptable carrier and any needed preservatives, buffers or propellants which can be required.

Actual dosage levels of active ingredients in the pharmaceutical compositions of this invention can be varied so as to obtain an amount of the active compound(s) which is effective to achieve the desired therapeutic response for a particular patient. The selected dosage level will depend upon the activity of the particular compound, the route of administration, the severity of the condition being treated and the condition and prior medical history of the patient being treated. However, it is within the skill of the art to start doses of the compound at levels lower than required to achieve the desired therapeutic effect and to gradually increase the dosage until the desired effect is achieved.

When used in the above or other treatments, a therapeutically effective amount of one of the inverse agonists of the present invention can be employed in pure form or, where such forms exist, in pharmaceutically acceptable salt, ester or prodrug form. Alternatively, the compound can be administered as a pharmaceutical composition containing the compound of interest in combination with one or more pharmaceutically acceptable excipients. The phrase “therapeutically effective amount” of the compound of the invention means a sufficient amount of the compound to treat disorders, at a reasonable benefit/risk ratio applicable to any medical treatment. It will be understood, however, that the total daily usage of the compounds and compositions of the present invention will be decided by the attending physician within the scope of sound medical judgement. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than required to achieve the desired therapeutic effect and to gradually increase the dosage until the desired effect is achieved.

The total daily dose of the inverse agonists of this invention administered to a human or lower animal may range from about 0.0001 to about 1000 mg/kg/day. For purposes of oral administration, more preferable doses can be in the range of from about 0.001 to about 5 mg/kg/day. If desired, the effective daily dose can be divided into multiple doses for purposes of administration; consequently, single dose compositions may contain such amounts or submultiples thereof to make up the daily dose.

The ability of compounds of the present invention to suppress appetite is described in detail hereinafter in the Examples. These Examples are presented to describe preferred embodiments and utilities of the invention and are not meant to limit the invention unless otherwise stated in the claims appended hereto.

### EXAMPLE 1

A histamine H₃ receptor binding analysis to determine the affinity of various imidazole derivatives for G-protein uncoupled receptors was performed as follows. Male Sprague-Dawley rats were purchased from Harlan Laboratories of Indianapolis, Ind. and housed two per cage on a 12 hour light/dark schedule with ad libitum access to Teklad Mouse/Rat Diet 7012 (available from Harlan Laboratories of Indianapolis, Ind.) and water in accordance with the Animal Welfare Act of 1994 and amendments. Animals were acclimated to laboratory conditions for a minimum of one week prior to tissue harvesting.

Histamine H₃ receptor affinity was determined in rat cerebral cortical membranes using the H₃ selective agonist ligand [³H]-N^{α}-methylhistamine ([³H]NAMHA, 78.9 Ci/mmole, available from NEN Research Products, Boston, Mass.) according to the method of West et al. in “Identification of Two H₃-Histamine Receptor Subtypes”, Mol. Pharmacol. 38: 610-613 (1990) as modified by Tedford et al. in “Development of trans-2-(1H-imidazol-4-yl) Cyclopropane Derivatives As New High-Affinity Histamine H₃ Receptor Ligands”, J. Pharmiacol. Exp. Ther. 289: 1160-1168 (1999).

Animals were anesthetized by NO₂ inhalation and euthanized by rapid decapitation. The cerebral cortical tissues were harvested and frozen on dry ice. Rat corticies were mechanically homogenized using a Tissue Tearer in 25 mM Tris buffer (pH 7.5 at 4° C.) containing: EDTA (10 mM), phenylmethylsulfonyl fluoride (0.1 mM), chymostatin and leupeptin (each 0.2 mg/50 mL). The homogenate was centrifuged in a Sorvall centrifuge at 40,000×g for 30 minutes. The pellet was re-suspended in 25 ml water and lysed on ice for 30 minutes. The homogenate was then re-centrifuged and the membrane lysis was repeated. The membranes were centrifuged and the final membrane pellets were resuspended in 14 volumes of water to yield approximately 200 μg protein/100 μl final concentration. The suspension was stored frozen at −80° C. prior to use. Protein concentrations were determined using the Coomassie Plus Protein Assay (available from Pierce of Rockford, Ill.).

Tables 1 and 2 show that certain of histamine H₃ receptor-binding compounds show increased affinity for receptor preparations that have been treated with GTPγS (guanosine 5′-o-(3-thiotriphosphate)). GTPγS causes dissociation of G-protein from receptors. Data are shown as percent of [³H]-N^{α}-methylhistamine bound (mean) for each table. The compound to be tested competes for binding with the radiolabelled compound [³H]-N^{α}-methylhistamine. When the compound to be tested binds preferentially to uncoupled receptors over coupled receptors, a lower percentage of binding will be measured for the radiolabelled histamine H₃ receptor ligand in the Tris-GTPγS treated system than in the Tris-treated system.

The results of Table 1 indicate that compound 1 binds to a GTPγS-treated cortical membrane preparation with higher apparent affinity than to a non-treated preparation. Conversely, certain histamine H₃ receptor-binding molecules, such as compound 2, show relatively little difference in binding to the two membrane preparations, as indicated by Table 2. A compound that shows preferred binding to a G-protein uncoupled receptor has been termed an “inverse agonist” (Milligan et al. “Inverse Agonism: Pharmacologicial Curiosity or Potential Therapeutic Strategy?” Trends Pharmacol. Sci. 16: 10-13 (1995) and “Inverse Agonism and the Regulation of Receptor Number” Trends Pharmacol. Sci. 18: 468-474 (1997)). Thus, molecules such as compound 1 can be classified as histamine H₃ receptor inverse agonists, whereas molecules such as compound 2 that have little preference for uncoupled or coupled receptors are referred to as antagonists.

The structures of the compounds tested are found below the relevant table. The compounds were prepared according to the procedures disclosed in WO 96/40126; WO 96/38142 and in U.S. Pat. Nos. 5,990,317; 6,008,240; and 5,486,526.

TABLE 1
Increased Binding Affinity of Compound 1 in the Presence of GTPγS.

| nM of compound | Tris | Tris-GTPγS |
|---|---|---|
| 10000 | −1.6 | 1.3 |
| 1000 | 12.6 | 4.00 |
| 100 | 67.9 | 23.8 |
| 30 | 88.4 | 42.6 |
| 10 | 102.5 | 76.4 |
| 3 | 101.6 | 93.3 |
| 1 | 95.7 | 98.3 |
| 0.3 | 108.1 | 102.8 |

Compound 1 = 4-((1R,2R)-trans-2-(O(2-cyclohexylethyl)
carboxamido)cyclopropyl)imidazole

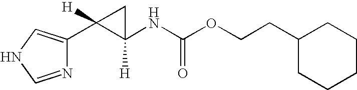

Compound 1

TABLE 2
GTPγS has a Negligible Effect on the Binding Affinity of
Compound 2.

| nM of compound | Tris | Tris-GTPγS |
|---|---|---|
| 1000 | 0.4 | 3.4 |
| 300 | 4.5 | 4.4 |
| 100 | 13.5 | 10.7 |
| 30 | 32.6 | 26.2 |
| 10 | 60.1 | 54.8 |
| 3 | 88.7 | 77.4 |
| 1 | 99.2 | 91.1 |
| 0.3 | 96.7 | 103.1 |
| 0.1 | 96.7 | 106.6 |
| 0.03 | 98.5 | 98.3 |

Compound 2 = (3Z)-4-(6-cyclohexylhex-3-en-1-yl)imidazole

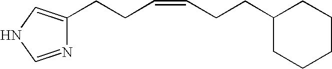

Compound 2

A variety of histamine H₃ receptor-binding compounds were also examined by the procedure described above for preferred binding to G-protein-uncoupled receptor preparations. The results of this analysis are summarized in Table 3, which lists the apparent binding affinities for GTPγS-treated and -untreated receptor preparations (the latter contains a mixture of coupled and uncoupled histamine H₃ receptors). Also shown in Table 3 are the ratios of binding affinities for the two membrane preparations. The higher the value for this ratio, the greater the binding affinity of the compound tested for G-protein-uncoupled receptors. Compounds having a ratio of at least two bind preferentially to uncoupled receptors.

TABLE 3
Calculated Binding Affinities to Histamine H₃ Receptor Preparations
in the Absence and Presence of GTPγS
Kᵢ(nM)

| | | | |
|---|---|---|---|
| Compound | (−) GTPγS | (+) GTPγS | Kᵢ Ratio (−/+) |

| | | | |
|---|---|---|---|
| 1 | 29.3 | 7.2 | 4.1 |
| 2 | 4.6 | 3.6 | 1.3 |
| 3 | 10.1 | 4.2 | 2.4 |
| 4 | 11.3 | 1.9 | 6.0 |
| 5 | 128.7 | 36.7 | 3.5 |
| 6 | 0.41 | 0.24 | 1.7 |
| 7 | 3.4 | 2.3 | 1.5 |
| 8 | 18.2 | 7.8 | 2.3 |
| 9 | 7.3 | 4.5 | 1.6 |

Compound 3 = 4-((1R,2R)-trans-2-(O-(2-cyclohexylmethyl)
carboxamido)cyclopropyl)imidazole

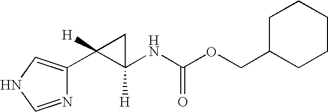

Compound 3
Compound 4 = 4-(((5S)(3Z)-5-amino-6-cyclohexyl)hex-3-en-1-
yl)imidazole

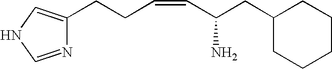

Compound 4
Compound 5 = 4-(N-(5-cyclohexylpentan-1-one-1-yl)piperidin-4-
yl)imidazole

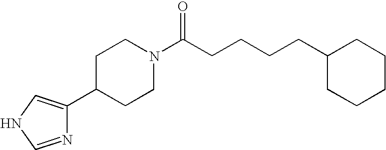

Compound 5
Compound 6 = (1R,2R)-trans-4-(2-(5,5-dimethylhex-1-ynyl)
cyclopropyl)imidazole

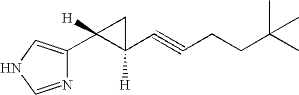

Compound 6
Compound 7 = 4-((1R,2R)-trans-2-(O-(2,6-dichlorophenylmethyl)
carboxamido)cyclopropyl)imidazole

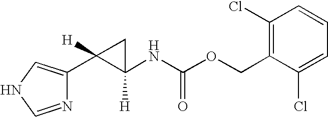

Compound 7
Compound 8 = 3-(1H-imidazol-4-yl)propyl-di(p-
fluorophenyl)-methylether

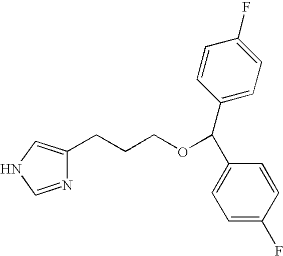

Compound 8
Compound 9 = 4-((1R,2R)-trans-2-(O-(3,3-dimethyl-but-1-yl)
carboxamido)cyclopropyl)imidazole

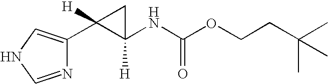

Compound 9

### EXAMPLE 2

The compounds summarized in Table 3 were examined for their ability to suppress food consumption by adult Sprague-Dawley rats. Male Sprague Dawley rats were purchased from Harlan Laboratories of Indianapolis, Ind. Rats were housed one per cage and maintained on a 12 hour light/dark schedule (lights-out at 12:30 p.m.) with ad libitum access to powdered Teklad Mouse/Rat Diet 7012 (available from Harlan Laboratories of Indianapolis, Ind.) and water. Rats were acclimated to laboratory conditions and food for 1 to 2 weeks prior to initiating food intake studies. On the day of experiments, test compounds were solubilized or suspended in vehicle/carrier and administered to rats (i.p. or p.o.) 30 to 60 minutes prior to the onset of the dark cycle. Food intake was determined at 1, 2, 4, and 24 hours following the onset of the dark cycle. Alternatively, rats were fasted (water provided) for 24 hours prior to evaluating the effects of compounds on food intake. For these studies, compounds or vehicle/carrier were administered and rats were placed in standard small animal metabolism chambers with ad libitum access to powdered Teklad Mouse/Rat Diet 7012 and water. Food intake was determined at 2 and 4 hours following the onset of feeding.

Example results are presented in Table 4. Data is cumulative food intake in grams, mean ± sem. In the table, * indicates a significant decrease in food intake compared to the vehicle-treated rats (t-test, p<0.05). A single administration of compound 3 caused a significant reduction of food consumption for up to 24 hours (Table 4).

TABLE 4
Compound 3 (10 mg/kg; i.p.) Causes a Significant
Reduction of Food Intake in Rats.

| | No treatment | Compound 3 |
|---|---|---|
| hr | mean (g) ± sem | mean (g) ± sem |
| 1 | 4.0 ± 0.3 | 1.8 ± 0.3* |
| 2 | 6.9 ± 0.5 | 4.4 ± 0.2* |
| 4 | 10.5 ± 0.6 | 7.1 ± 0.5* |
| 24 | 28.9 ± 2.3 | 17.4 ± 1.2* |

The percent reduction in food intake by rats two hours after intraperitoneal administration of each compound is shown in Table 5. As can be seen, some of the compounds (i.e., 1,3-5, 8) caused a statistically significant reduction of food consumption. Conversely, compounds 2, 6, 7 and 9 had no measurable effect on the amount of food consumed by the rats. Interestingly, it appears that compounds with approximately 2-fold greater affinity for GTPγS-treated receptor samples than for non-treated preparations cause appetite suppressing effects, whereas compounds falling below this ratio do not cause appreciable changes in food consumption (see Tables 3 and 5). Thus, inverse agonists cause significant reduction in food consumption, whereas antagonists do not. In Table 5, * indicates a significant decrease in food intake compared to the vehicle-treated rats (t-test, p<0.05). The ex vivo binding values in Table 5 represent the drug dose required to obtain ½-maximal H₃ receptor occupancy in the brain following intraperitoneal administration. The dose values shown in Table 5 represent the dosage of drug utilized during the food intake studies. In all cases, the tested dose exceeded the amount needed to obtain ½-maximal H₃ receptor occupancy in the brain.

TABLE 5
Effects of Selected Compounds on Food Intake Two Hours Following
the Onset of the Dark Cycle (% Decrease in Food Intake)

| | | | |
|---|---|---|---|
| | | Decrease in | |
| | | Food Intake | ″EX Vivo Binding |
| Compound | Dose | (%) | (mg/kg) |

| | | | |
|---|---|---|---|
| 1 | 10 | 32.6 ± 4.3* | 31 ± 1.3 |
| 2 | 3 | 5.5 ± 0.8 | 0.92 ± 0.01 |
| 3 | 10 | 36.4 ± 3.3* | 2.6 ± 1.3 |
| 4 | 20 | 53.9 ± 11.1* | ˜15 |
| 5 | 10 | 67.6 ± 9.6* | 8.0 ± 1.3 |
| 6 | 1 | −4.6 ± 0.8 | 0.08 ± 0.03 |
| 7 | 10 | 0.8 ± 0.0 | 2.9 ± 1.3 |
| 8 | 10 | 56.6 ± 8.3* | 5.6 ± 0.12 |
| 9 | 10 | −25.1 ± 3.8 | 3.7 ± 0.4 |

All references cited are hereby incorporated by reference.

The present invention is illustrated by way of the foregoing description and examples. The foregoing description is intended as a non-limiting illustration, since many variations will become apparent to those skilled in the art in view thereof. It is intended that all such variations within the scope and spirit of the appended claims be embraced thereby.

Changes can be made in the composition, operation and arrangement of the method of the present invention described herein without departing from the concept and scope of the invention as defined in the following claims:

We claim:

1. A method for promoting weight loss and treating eating disorders comprising administering to a patient in need of such weight loss or treatment an effective amount of an inverse agonist of histamine H₃ receptors, with the proviso that said inverse agonist is not thioperamide.

2. The method of claim 1 wherein said inverse agonist is selected from the group consisting of 4-{(1R,2R)-trans-2-[O-(2-cyclohexylethyl) carboxamido]cyclopropyl}imidazole, 4-{(1R,2R)-trans-2-[O-(2-cyclohexylmethyl)carboxamido]cyclopropyl}imidazole and 3-(1H-imidazol-4-yl)propyl-di(p-fluorophenyl)-methyl ether.

3. The method of claim 1 wherein said inverse agonist is administered by intravenous, intramuscular, intraperitoneal or subcutaneous injection.

4. The method of claim 1 wherein said inverse agonist is administered orally.

5. The method of claim 1 wherein from about 0.01 mg/kg to about 200 mg/kg of said inverse agonist is administered in a single dose or divided dose per day.
